(19) United States
(12) Patent Application Publication
(10) Pub. No.: US 2012/0136005 A1
(43) Pub. Date: May 31, 2012

(54) ELTOPRAZINE FOR THE TREATMENT OF ANXIETY

(75) Inventors: Barbara Valastro, Frankfurt Am Main (DE); Lutz Franke, Bad Vilbel (DE); Andreas Gravius, Partenstein (DE)

(73) Assignee: MERZ PHARMA GmbH & CO. KGaA, Frankfurt am Main (DE)

(21) Appl. No.: 13/379,426

(22) PCT Filed: Jun. 30, 2010
(86) PCT No.: PCT/EP2010/003966

§ 371 Date: Feb. 17, 2012

Related U.S. Application Data
(60) Provisional application No. 61/269,861, filed on Jun. 30, 2009.

(51) Int. Cl. A61K 31/496; A61P 25/24; A61P 25/22

(52) U.S. Cl. 514/254.11

(57) ABSTRACT

The present invention relates to the treatment of an individual afflicted with certain forms of anxiety, the instant treatment comprising administering to the individual an effective amount of eltoprazine or a pharmaceutically acceptable salt thereof

### FIELD OF THE INVENTION

The present invention relates to the efficient treatment of an individual afflicted with certain forms of anxiety, the instant treatment comprising administering to the individual an effective amount of eltoprazine or a pharmaceutically acceptable salt thereof.

### BACKGROUND OF THE INVENTION

This invention relates to an innovative method of treating patients afflicted with certain forms of anxiety.

Eltoprazine (1-(2,3-dihydro-1,4-benzodioxin-8-yl)piperazine) was synthesized more than two decades ago by scientists at Duphar International Research B. V., Netherlands, and was first described in EP 0138280 (published Apr. 24, 1985) as intermediate for the synthesis of piperazine derivatives. European patent application EP 0189612 (filed Dec. 16, 1985) disclosed for the first time the use of eltoprazine as a pharmaceutical compound. Eltoprazine and related compounds were characterized as compounds having psychotropic activity that could be used, for example, for treating aggressive behavior, and ED50 values for a mouse model for fighting behavior were given in EP 0189612.

The application mentioned further indications including other affections and diseases caused by disturbances in the central nervous system, such as psychoses, fear, depression, or the use as analgetics. Furthermore, the use of such compounds for treating hematological disorders was discussed based on the thrombolytic activity of the compounds. However, no data were given.

Subsequently, the use of eltoprazine in the treatment of aggressive behaviour was tested in several clinical trials, for example in a trial of eltoprazine in the treatment of aggressive behavior in patients with epilepsy or Gilles de la Tourette's Syndrome (see Moriarty et al., Human Psychopharmacology: Clinical and Experimental, 9 (1994) 253-258), or in aggressive mentally handicapped patients (see de Koning et al., Int Clin Psychopharmacol. 9 (1994) 187-94). While these clinical trials did not result in an approval of eltoprazine in these indications, eltoprazine did prove to be clinically safe (de Koning, loc. cit.).

Recently, eltoprazine has been studied in a phase IIa clinical trial for the treatment of ADHD patients (see US 2009/0104261; http://www.psychogenics.com/pdf/Psychogenics%20-%20Eltoprazine%20Press%20Release%20_Jun%2008.pdf).

Anxiety in general is a feeling of unease. In more severe and excessive forms, anxiety can result in various anxiety disorders that require medical and/or psychological attention, such as phobias, obsessive-compulsive disorder (OCD), panic disorder, post-traumatic stress disorder (PTSD), or generalised anxiety disorder (GAD). Anxiety is closely related to the serotonergic system.

The present invention relates to the use of eltoprazine and its salts, solvates and conjugates, which we have determined possesses a unique receptor profile. Consequently, the present invention relates to the use of eltoprazine for the treatment of certain forms of anxiety.

### SUMMARY OF THE INVENTION

The present invention relates to a method of treating or preventing an anxiety disorder in a subject in need thereof, comprising administering an effective amount of eltoprazine or a pharmaceutically acceptable salt thereof, wherein such disorder is characterized by a down-regulation of 5-HT1a receptor and/or 5-HT1a receptor activity.

A further aspect of the invention relates to such a method comprising administering an effective amount of eltoprazine hydrochloride.

A further aspect of the invention relates to such a method wherein eltoprazine is administered in a range from about 5 mg to about 75 mg/day.

A further aspect of the invention relates to such a method wherein eltoprazine is administered in a range from about 5 mg to about 50 mg/day.

A further aspect of the invention relates to such a method wherein eltoprazine is administered in a range from about 5 mg to about 60 mg/day.

A further aspect of the invention relates to such a method wherein eltoprazine is administered in a range from about 5 mg to about 40 mg/day.

A further aspect of the invention relates to such a method wherein eltoprazine is administered in a range from about 5 mg to about 20 mg/day.

A further aspect of the invention relates to such a method wherein eltoprazine is administered at about 20 mg/day.

A further aspect of the invention relates to such a method wherein eltoprazine is administered at about 40 mg/day.

A further aspect of the invention relates to such a method wherein eltoprazine is administered at about 60 mg/day.

A further aspect of the invention relates to such a method wherein eltoprazine is administered between about 10 and 15 mg/day.

A further aspect of the invention relates to such a method wherein eltoprazine or a pharmaceutically acceptable salt thereof is administered once a day, twice a day (b.i.d.), or three times a day.

A further aspect of the invention relates to such a method wherein eltoprazine is administered in an oral formulation.

A further aspect of the invention relates to such a method wherein the subject suffers from posttraumatic stress disorder.

A further aspect of the invention relates to a composition comprising eltoprazine or a pharmaceutically acceptable salt thereof (e.g., eltoprazine hydrochloride) for the treatment or prevention of an anxiety disorder, wherein such disorder is characterized by a down-regulation of 5-HT1a receptor and/or 5-HT1a receptor activity.

A further aspect of the invention relates to the use of eltoprazine or a pharmaceutically acceptable salt thereof (e.g., eltoprazine hydrochloride) for the manufacture of a medicament for the treatment or prevention of an anxiety disorder, wherein such disorder is characterized by a down-regulation of 5-HT1a receptor and/or 5-HT1a receptor activity.

A further aspect of the invention relates to the above-defined composition or use wherein eltoprazine or a pharmaceutically acceptable salt thereof (e.g., eltoprazine hydrochloride) is for administration in a range from about 5 mg to about 150 mg/day, or in a range from about 5 mg to about 100 mg/day, or in a range from about 5 mg to about 75 mg/day, in a range from about 5 mg to about 75 mg/day, or in a range from about 5 mg to about 60 mg/day, or in a range from about 5 mg to about 50 mg/day, or in a range from about 5 mg to about 40 mg/day, or in a range from about 5 mg to about 20 mg/day, or in a range from about 5 mg to about 15 mg/day, or wherein eltoprazine is for administration at about 10 mg/day, wherein eltoprazine is for administration at about 15 mg/day, is for administration at about 20 mg/day, or eltoprazine is for administration at about 40 mg/day, or eltoprazine is for administration at about 60 mg/day, or eltoprazine is for administration at about 80 mg/day.

A further aspect of the invention relates to the above-defined composition or use wherein eltoprazine or a pharmaceutically acceptable salt thereof (e.g., eltoprazine hydrochloride) is for administration once a day, twice a day (b.i.d.), or three times a day.

A further aspect of the invention relates to the above-defined composition or use wherein eltoprazine or a pharmaceutically acceptable salt thereof (e.g., eltoprazine hydrochloride) is for administration in an oral formulation.

A further aspect of the invention relates to the above-defined composition or use wherein the subject to be treated suffers from an anxiety disorder, wherein such disorder is characterized by a down-regulation of 5-HT1a receptor and/or 5-HT1a receptor activity.

A further aspect of the invention relates to the above-defined composition or use wherein the anxiety disorder is posttraumatic stress disorder.

A further aspect of the invention relates to a method comprising administering a therapeutically effective amount of eltoprazine or a pharmaceutically acceptable salt thereof in combination with at least one additional pharmaceutical agent which has been shown to be effective for the treatment of such anxiety disorder.

### BRIEF DESCRIPTION OF THE DRAWINGS

FIG. 1 shows the potency of eltoprazine on different serotonin receptors at relevant plasma concentration (<0.5 μM).

FIG. 2 shows the effect of eltoprazine on AIM scores in unilaterally 6-OHDA-lesioned rats.

FIG. 3 shows the effect of eltoprazine on turning behavior induced by high dose of L-DOPA in unilaterally 6-OHDA-lesioned rats.

FIG. 4 illustrates the effect of an acute treatment with eltoprazine on percent time spent freezing during re-exposure to the conditioning context. The data are expressed as mean±SEM. *p<0.05 vs vehicle calculated by ANOVA on ranks followed by rank sum tests (n=8−9).

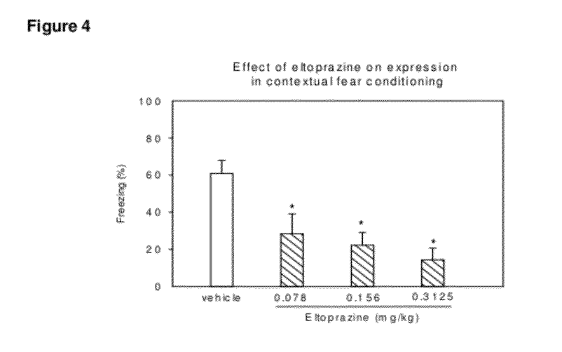

FIG. 5 illustrates the effect of an acute treatment with eltoprazine on locomotor activity in the open field. The data are expressed as mean±SEM. *p<0.05 vs vehicle calculated by ANOVA followed by Dunnett's test (n=8).

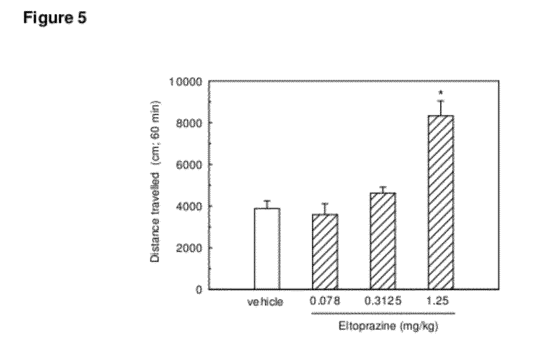

FIG. 6 illustrates the effect of an acute treatment with eltoprazine on percent time spent in the open arms of the elevated plus maze. The data are expressed as mean±SEM. *p<0.05 vs vehicle calculated by ANOVA followed by Dunnett's test (n=8).

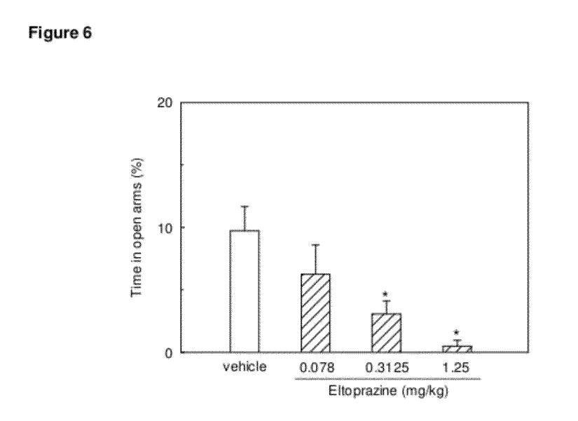

FIG. 7 illustrates the effect of an acute treatment with eltoprazine on number of shocks received in the shock-suppressed drinking (Vogel) test. The data are expressed as mean±SEM (n=12).

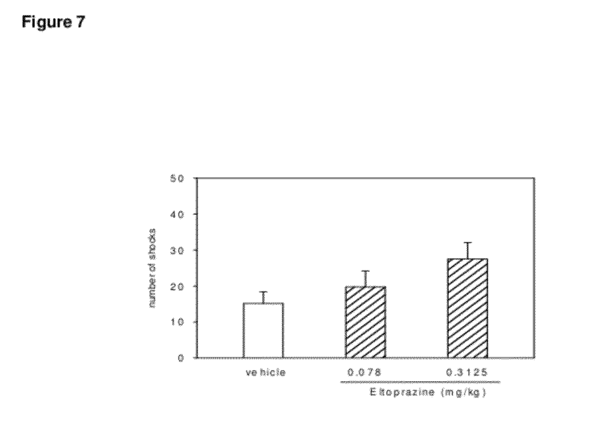

FIG. 8 illustrates the effect of an acute treatment with eltoprazine on shock sensitivity in the shock-suppressed drinking (Vogel) test. The data are expressed as mean±SEM. *p<0.05 vs vehicle calculated by ANOVA followed by Dunnett's test (n=12).

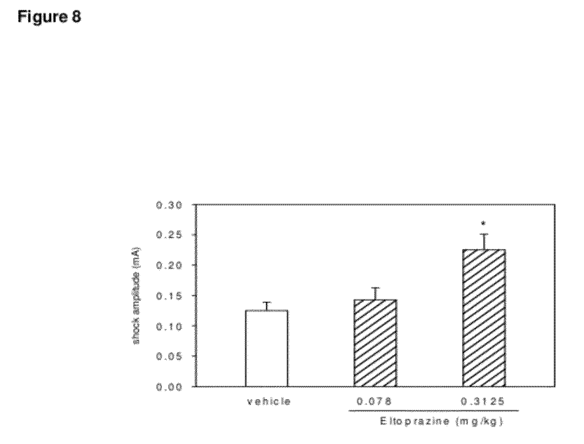

### DETAILED DESCRIPTION OF THE INVENTION

The peculiarity of this invention compared to former treatment approaches for an anxiety disorder, wherein such disorder is characterized by a down-regulation of 5-HT1a receptor and/or 5-HT1a receptor activity, is the so far unknown therapeutic efficiency of eltoprazine, which is based on the unique receptor profile with several components potentiating each other in an unexpected way, as evidenced by the surprising activity of eltoprazine in a L-DOPA-induced dyskinesia model.

In the past, the pharmacological action of eltoprazine was mainly attributed to its agonistic effect on the 5-HT1a and 5-HT1b receptors (see Schipper J, Tulp M T M, Sijbesma H. Neurochemical profile of eltoprazine. Drug metabolism and Drug interactions 1990 8:85-114), while its effect on 5-HT2c (initially designated 5-HT1c) was characterized as antagonistic.

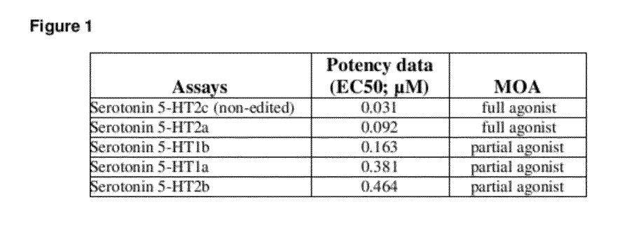

However, by using recombinant cell lines expressing the 5-HT1a, 5-HT-1b, 5-HT2a, 5-HT2b, 5-HT2c edited (data not shown) and 5-HT2c non edited (ne) recombinant receptors in assays for testing the functional activity of eltoprazine, we have surprisingly found that eltoprazine acts primarily as a full agonist at the 5-HT2a and 5-HT2c receptors in human recombinant cell lines, in addition to its partial agonistic action on 5-HT1a and 5-HT1b receptors (see FIG. 1).

Several anxiety disorders, including posttraumatic stress disorder, have been characterized as including a downregulation of the 5-HT1a receptor (see van Praag, Prog Neuropsychopharmacol Biol Psychiatry. 28 (2004) 923-35). One of the approaches taken for treating posttraumatic stress disorder thus focuses on the application of 5-HT2 agonists, such as MDMA (ecstasy), which has been studied in phase II clinical trials for the treatment of PTSD.

The present invention relates to the use of eltoprazine and its salts, solvates and conjugates, which possesses a unique receptor profile targeting the 5-HT2a, 5-HT2c, 5-HT1a and 5-HT1b receptors at relevant plasma concentration. Thus, eltoprazine with its unique receptor profile should be ideally suited for addressing anxiety disorders such as posttraumatic stress disorder. In theory, the unique efficacy could be achieved also through a cocktail of drugs mimicking these composite effects, however, such a cocktail of several molecules cannot successfully be developed in a clinical setting obeying regulatory requirements.

As used herein, the term “subject” encompasses mammals including animals and humans.

The term eltoprazine is known in the art and may also be known as DU-28853 and 1-(2,3-dihydro-1,4-benzodioxin-5-yl)piperazine. As used herein, eltoprazine refers to the substance, as well as its pharmaceutically acceptable salts.

The term “agonist” refers to a substance that binds to a receptor and mimics the cellular effect of the native or endogenous ligand for the same receptor. The term agonist includes the class of agents called full agonists, which bind and display full efficacy at the receptor, and partial agonists, which have only partial efficacy at the receptor. Partial agonists may also be seen as competitive antagonists, competing away the endogenous ligand when it is in excess or give a sub maximal response when inadequate amount of endogenous ligand is present. The term “activation” refers to the state of a receptor when an agonist is bound to it.

The term “treat” is used herein to mean to relieve or alleviate at least one symptom of a disease in a subject. Within the meaning of the present invention, the term “treat” also denotes to arrest, delay the onset (i.e., the period prior to clinical manifestation of a disease) and/or reduce the risk of developing or worsening a disease.

The term “therapeutically effective” applied to dose or amount refers to that quantity of a compound or pharmaceutical composition sufficient to result in a desired activity upon administration to a mammal in need thereof.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). The term “pharmaceutically acceptable” may also mean approved by a regulatory agency of the Federal or a state government or listed in the U.S. Pharmacopeia or other generally recognized pharmacopeia for use in mammals, and more particularly in humans.

The term “salt” is defined as a chemical containing different charged components. The term salt also includes hydrates and solvates. Contemplated in the instant description are pharmaceutically acceptable salts, which salts may include, but are not limited to, acid addition salts, such as those made with hydrochloric, sulphuric, nitric, phosphoric, acetic, maleic, fumaric, tartaric, citric, benzoic, methane sulphonic, naphthalene sulphonic, p-toluene sulphonic acid. All of these salts (or other similar salts) may be prepared by conventional means. The nature of the salt is not critical, provided that it is non-toxic and does not substantially interfere with the desired pharmacological activity.

Eltoprazine may be used according to the invention in the form of any of pharmaceutically acceptable salts, solvates and conjugates. Any references to eltoprazine in this description should be understood as also referring to such salts, solvates and conjugates.

The term “carrier” applied to pharmaceutical compositions of the invention refers to a diluent, excipient, or vehicle with which an active compound (e.g., eltoprazine) is administered. Such pharmaceutical carriers may be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by A. R. Gennaro, 20^{th} Edition.

The term “about” or “approximately” usually means within 20%, alternatively within 10%, including within 5% of a given value or range. Alternatively, especially in biological systems, the term “about” means within about a log (i.e., an order of magnitude), including within a factor of two of a given value.

In conjunction with the methods of the present invention, also provided are pharmaceutical compositions comprising a therapeutically effective amount of eltoprazine. The compositions of the invention may further comprise a carrier or excipient (all pharmaceutically acceptable). The compositions may be formulated e.g. for once-a-day administration, twice-a-day administration, or three times a day administration.

The active ingredient (e.g., eltoprazine) or the composition of the present invention may be used for the treatment of at least one of the mentioned disorders, wherein the treatment is adapted to or appropriately prepared for a specific administration as disclosed herein (e.g., to once-a-day, twice-a-day, or three times a day administration). For this purpose the package leaflet and/or the patient information contains corresponding information.

In another embodiment, a therapeutically effective amount of eltoprazine is administered twice per day. In particular, eltoprazine is administered once in the morning, and once in the middle of the day, particularly at about lunchtime, wherein the lunchtime treatment is between about 6 and 10, particularly between about 7 and 9 hours before the patient wishes to go to bed. Such an administration should allow for the eltoprazine plasma level to ebb away during evening and at nighttime, reducing the risk of an impaired REM-(rapid eye movement)-activity.

In certain such embodiments, the treatment is the treatment of posttraumatic stress disorder.

In another embodiment, the first dosage of eltoprazine consist of about 55 to 65% of the total daily dosage amount, and the second dose of eltoprazine comprises the remaining total daily dosage amount.

The active ingredient (e.g., eltoprazine) or the composition of the present invention may be used for the manufacture of a medicament for the treatment of at least one of the mentioned disorders, wherein the medicament is adapted to or appropriately prepared for a specific administration as disclosed herein (e.g., to once-a-day, twice-a-day, or three times a day administration). For this purpose the package leaflet and/or the patient information contains corresponding information.

According to the present invention, the dosage form of eltoprazine, or an eltoprazine salt, may be a solid, semisolid, or liquid formulation according to the following.

Eltoprazine may be administered orally, topically, parenterally, or mucosally (e.g., buccally, by inhalation, or rectally) in dosage unit formulations containing conventional non-toxic pharmaceutically acceptable carriers. In another embodiment, eltoprazine may be formulated as a flavored liquid (e.g., peppermint flavor). Eltoprazine may be administered orally in the form of a capsule, a tablet, granules, pellets or the like, or as a semi-solid, or liquid formulation (see Remington's Pharmaceutical Sciences, 20^{th} Edition, by A. R. Gennaro).

For oral administration in the form of a tablet or capsule, eltoprazine may be combined with non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like.

The tablets may be coated with a concentrated sugar solution which may contain e.g., gum arabic, gelatine, talcum, titanium dioxide, and the like. Alternatively, the tablets may be coated with a polymer that dissolves in a readily volatile organic solvent or mixture of organic solvents. In specific embodiments, eltoprazine is formulated in immediate-release (IR) or modified-release (MR) tablets. Immediate release solid dosage forms permit the release of most or all of the active ingredient over a short period of time, such as 60 minutes or less, and make rapid absorption of the drug possible. Modified release solid oral dosage forms permit the sustained release of the active ingredient over an extended period of time in an effort to maintain therapeutically effective plasma levels over similarly extended time intervals and/or to modify other pharmacokinetic properties of the active ingredient. For example, eltoprazine may be formulated in a modified release dosage form (including modified release tablets) to provide a dose of eltoprazine.

For the formulation of soft gelatin capsules, eltoprazine may be admixed with e.g., a vegetable oil or polyethyleneglycol. Hard gelatin capsules may contain granules of the active substances using either the above-mentioned excipients for tablets e.g., lactose, saccharose, sorbitol, mannitol, starches (e.g., potato starch, corn starch or amylopectin), cellulose derivatives or gelatine. Also liquids or semisolids of the drug may be filled into hard gelatine capsules.

Eltoprazine may also be introduced in microspheres or microcapsules, e.g., fabricated from polyglycolic acid/lactic acid (PGLA) (see, e.g., U.S. Pat. Nos. 5,814,344; 5,100,669 and 4,849,222; PCT Publications No. WO 95/11010 and WO 93/07861). Biocompatible polymers may be used in achieving controlled release of a drug, include for example, polylactic acid, polyglycolic acid, copolymers of polylactic and polyglycolic acid, poly(epsilon-caprolactone), polyhydroxybutyric acid, polyorthoesters, polyacetals, polyhydropyrans, polycyanoacrylates, and cross-linked or amphipathic block copolymers of hydrogels.

Formulation of eltoprazine in a semi-solid or liquid form may also be used. Eltoprazine may constitute between 0.1 and 99% by weight of the formulation, more specifically between 0.5 and 20% by weight for formulations intended for injection and between 0.2 and 50% by weight for formulations suitable for oral administration.

In one embodiment of the invention, eltoprazine is administered in a modified release formulation. Modified release dosage forms provide a means for improving patient compliance and for ensuring effective and safe therapy by reducing the incidence of adverse drug reactions. Compared to immediate release dosage forms, modified release dosage forms may be used to prolong pharmacologic action after administration, and to reduce variability in the plasma concentration of a drug throughout the dosage interval, thereby eliminating or reducing sharp peaks.

A modified release form dosage may comprise a core either coated with or containing a drug. The core is then coated with a release-modifying polymer within which the drug is dispersed. The release-modifying polymer disintegrates gradually, releasing the drug over time. Thus, the outer-most layer of the composition effectively slows down and thereby regulates the diffusion of the drug across the coating layer when the composition is exposed to an aqueous environment, i.e. the gastrointestinal tract. The net rate of diffusion of the drug is mainly dependent on the ability of the gastric fluid to penetrate the coating layer or matrix and on the solubility of the drug itself.

In another embodiment of the invention, eltoprazine is formulated in an oral, liquid formulation. Liquid preparations for oral administration may take the form of, for example, solutions, syrups, emulsions or suspensions, or they may be presented as a dry product for reconstitution with water or other suitable vehicle before use. Preparations for oral administration may be suitably formulated to give controlled or postponed release of the active compound.

For oral administration in liquid form, eltoprazine may be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) may also be added to stabilize the dosage forms. For example, solutions may contain from about 0.2% to about 20% by weight of eltoprazine, with the balance being sugar and mixture of ethanol, water, glycerol and propylene glycol. Optionally, such liquid formulations may contain coloring agents, flavoring agents, saccharine and carboxymethyl-cellulose as a thickening agent or other excipients.

In another embodiment, a therapeutically effective amount of eltoprazine is administered in an oral solution containing a preservative, a sweetener, a solubilizer, and a solvent. The oral solution may include one or more buffers, flavorings, or additional excipients. In a further embodiment, a peppermint or other flavoring is added to the eltoprazine oral liquid formulation.

For administration by inhalation, eltoprazine may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Solutions for parenteral applications by injection may be prepared in an aqueous solution of the compound or of a water-soluble pharmaceutically acceptable salt of the active substances, for example in a concentration of from about 0.5% to about 10% by weight. These solutions may also contain stabilizing agents and/or buffering agents and may conveniently be provided in various dosage unit ampoules.

The formulations of the invention may be delivered parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion. Formulations for injection may be presented in unit dosage form, e.g., in ampoules or in multi-dose containers, with an added preservative. Alternatively, the active ingredient may be in powder form for reconstitution with a suitable vehicle, e.g., sterile pyrogen-free water, before use.

The invention also provides a pharmaceutical pack or kit comprising one or more containers containing eltoprazine and, optionally, more of the ingredients of the formulation. In a specific embodiment, eltoprazine is provided as an oral solution for administration with the use of a 2-teaspoon capacity syringe (dosage KORC®). Each oral syringe has hatch marks for measurement, with lines on the right side of the syringe (tip down) representing tsp units, and those on the left representing ml units.

The optimal therapeutically effective amount may be determined experimentally, taking into consideration the exact mode of administration, form in which the drug is administered, the indication toward which the administration is directed, the subject involved (e.g., body weight, health, age, sex, etc.), and the preference and experience of the physician or veterinarian in charge.

In certain embodiments, the invention relates to the use of eltoprazine in the preventive treatment of a patient an anxiety disorder, wherein such disorder is characterized by a down-regulation of 5-HT1a receptor and/or 5-HT1a receptor activity.

Dosage units for rectal application may be solutions or suspensions or may be prepared in the form of suppositories or retention enemas comprising eltoprazine in a mixture with a neutral fatty base, or gelatin rectal capsules comprising the active substances in admixture with vegetable oil or paraffin oil.

Toxicity and therapeutic efficacy of the compositions of the invention may be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it may be expressed as the ratio LD₅₀/ED₅₀. Compositions that exhibit large therapeutic indices are preferred.

Suitable daily doses of the active ingredient of the invention (eltoprazine) in therapeutic treatment of humans are within the range from about 5 mg to about 150 mg per day, such as from about 5 mg to about 120 mg, from about 5 mg to about 100 mg, or from about 5 mg to about 75 mg, or from about 5 mg to about 60 mg, or from about 5 mg to about 50 mg, or from about 10 mg to about 20 mg, or from about 10 mg to about 15 mg, such as 10 mg or 15 mg or 20 mg or 30 mg or 40 mg or 60 mg or 80 mg, per day. For example the daily dose may be body weight-adjusted such as 40 mg/day up to 80 kg body weight or 60 mg/day for patients with a body weight of 80 kg. In modified release formulations the amounts of active ingredient per day could also be higher due to reduced bioavailability, e.g. up to 200 mg/day. An equimolar amount of another pharmaceutically acceptable salt, a solvate, a conjugate or a derivative thereof, such as eltoprazine hydrochloride, is also suitable. For pediatric subjects aged 4-14, eltoprazine may be administered as an oral, liquid dosage form, at reduced amounts, for example from about 1 mg/day, up to about 5 mg/day.

In particular embodiments, the daily dosage of eltoprazine is between about 10 and 20 mg/day.

The daily doses indicated herein may be administered, for example, as one or two dosing units once, twice or three times per day. Suitable doses per dosage unit may therefore be the daily dose divided (for example, equally) between the number of dosage units administered per day, and will thus typically be about equal to the daily dose or one half, one third, one quarter or one sixth thereof. Dosages per dosage unit may thus be calculated from each daily dosage indicated herein. A daily dose of 5 mg, for example may be seen as providing a dose per dosage unit of, for example, about 5 mg, 2.5 mg, 1.67 mg, 1.25 mg and 0.83 mg, depending upon the dosing regimen chosen. Correspondingly, a dosage of 50 mg per day corresponds to dosages per dosing unit of, for example, about 50 mg, 25 mg, 16.7 mg, 12.5 mg, and 8.33 mg for corresponding dosing regimens. In other embodiments, especially in cases where only two daily doses are administered, an unequal split of the first and the second dosage is envisaged. In such cases, for example, the first dosage comprises about 55 to 65% of the total daily dosage. For example, a daily dosage of 10 mg is split into a first dosage of 6 mg and a second dosage of 4 mg. In cases, in which more than two administration per day are envisaged, the dosage might be split also unequally, wherein the second and further dosages are reduced in comparison to the dosage before. For example, if three dosages per day are administered, the first dosage could be about one half of the total daily dosage, i.e. between about 40% to 60%, the second dosage could be about one third of the total daily dosage, i.e. between about 20% to 40%, and the third dosage could be about one sixth of the total daily dosage, i.e. between about 5% to 20%. For example, a daily dosage of 10 mg could be split into a first dosage of 6 mg, a second dosage of 3 mg and a third dosage of 1 mg.

Treatment duration may be short-term, e.g., several weeks (for example 8-14 weeks), or long-term until the attending physician deems further administration no longer is necessary.

Eltoprazine may be administered as a single agent or in combination with another agent.

The term “combination” applied to active ingredients is used herein to define two separate pharmaceutical compositions, each comprising an active agent (e.g. eltoprazine, and another pharmaceutical composition comprising another agent prescribed for such disorder), to be administered conjointly.

Within the meaning of the present invention, the term “conjoint administration” is used to refer to administration of eltoprazine, and a second active agent simultaneously in different compositions, or sequentially. For the sequential administration to be considered “conjoint”, however, eltoprazine, and the second active agent must be administered separated by a time interval, which still permits the resultant beneficial effect for treating such disorder in a mammal.

### EXAMPLES

The following examples illustrate the invention without limiting its scope.

### Example 1

### Neurochemical Profile of Eltoprazine

### 5-HT1a and 5-HT1b Receptors

Eltoprazine was tested for activity on the human serotonin 5-HT1a and 5-HT1b receptors using a GTPγS assay. Recombinant membranes obtained from CHO-K1 cells expressing either the 5-HT1a or 5-HT1b receptors were mixed with GDP (volume:volume) and incubated for at least 15 min on ice. In parallel, GTPγ[35S] was mixed with the beads (volume:volume) just before starting the reaction. The following reagents were successively added in the wells of an Optiplate (Perkin Elmer): 50 μl of test compound, 20 μl of the membranes:GDP mix, 10 μl of assay buffer (for agonist testing) and 20 μl of the GTPγ[35S]:beads mix. The plates were covered with a top seal, shaken on an orbital shaker for 2 min, and then incubated for 1 h at room temperature. Then the plates were centrifuged for 10 min at 2000 rpm, incubated at room temperature 1 h and counted for 1 min/well with a PerkinElmer TopCount reader.

### 5-HT2a, 5-HT2b and 5-HT2c Non-Edited Receptor

Aequorin cell lines expressing the 5-HT2a, 5-HT2b and 5-HT2c non-edited (ne) recombinant receptors were used to evaluate the functional activity of eltoprazine. Aequorin cells grown 18 h prior to the test in media without antibiotics were detached by gentle flushing with PBS-EDTA (5 mM EDTA), recovered by centrifugation and resuspended in “assay buffer” (DMEM/HAM's F12 with HEPES+0.1% BSA protease free). Cells were incubated at room temperature for at least 4 h with Coelenterazine h (Molecular Probes). The reference agonist used were 5-HT and α-methyl-5-HT. For agonist testing, 50 μl of cell suspension were injected on 50 μl of test compound or reference agonist plated in a 96-well plate. The resulting emission of light was recorded using the Hamamatsu Functional Drug Screening System 6000 (FDSS 6000).

### Results

FIG. 1 illustrates the potency of eltoprazine on different serotoninergic receptor activated at relevant plasma concentration.

### Example 2

### Effect of Eltoprazine on L-DOPA Induced Dyskinesia in the 6-Hydroxydopamine (6-OHDA)-Lesioned Rat Model of Parkinson's Disease

### Materials and Methods

### Animals

Male Sprague Dawley rats (Elevage Janvier, Le Genest Saint Isle, France) weighing between 220 and 250 g at the beginning of the study are used in these experiments. They are housed under a 12 h light/dark cycle with free access to standard pelleted food and tap water. Animal treatment and experimental procedures are approved by local ethical committees (Regierungspräsidium Darmstadt; Germany).

### Dopamine-Denervating Lesions

Dopamine-denervating lesions are performed on rats anaesthetized with a 5:1 mixture of ketamine and xylazine (1 ml/kg, i.p.). All rats receive unilateral injection of 6-hydroxydopamine (6-OHDA-HCl) (3 μg/μl in 0.02% ascorbate-saline) into the right ascending DA fibre bundle at the following coordinates (in mm relative to bregma and the dural surface): (1) A=−4.4, L=−1.2, V=−7.8, tooth bar −2.3 (7.5 μg deposit); (2) A=−4.0, L=−0.75, V=−8.0, tooth bar=+3.4 (6 μg deposit). Injections are performed at the rate of 1 μl/min (allowing an additional 3 min before retracting the needle) using a 10 μl Hamilton microsyringe with a 26-gauge steel cannula. In order to assess the efficacy of the lesions, all rats are tested for amphetamine-induced rotation 2 weeks after the 6-OHDA injections. The animals' turning behaviour is recorded in an automated rotometer (TSE Rotameter System, TSE-Systems GmbH, Bad Homburg, Germany) over a 90 min period after the intraperitoneal (i.p.) injection of 2.5 mg/kg dexamphetamine sulphate dissolved in saline. Only the rats showing rotational scores>5 net full turns/min in the direction ipsilateral to the lesion are selected for the study.

### Experimental Design and Drug Treatment

At 6-8 weeks post-lesion, rats are treated for 21 days with a single daily i.p. injection of 6 mg/kg of L-DOPA mixed with 15 mg/kg of the peripheral DOPA-decarboxylase inhibitor benserazide hydrochloride or with saline (vehicle controls). L-DOPA and benserazide are dissolved in a physiological saline solution. Chronic treatment with this dose of L-DOPA has been shown to induce gradual development of dyskinetic-like movements in 6-OHDA-lesioned rats. After approx. 3 weeks of the daily treatment, rats are injected 30 min before the evaluation of abnormal involuntary movement (AIM)s with different doses of eltoprazine hydrochloride (0.08, 0.3, 1.25 and 5 mg/kg, s.c.), followed by L-DOPA (L-DOPA 6 mg/kg, and benserazide 15 mg/kg), i.p., 10 min before the beginning of the test.

### Behavioral Test

In order to evaluate the severity of LID, AIMs are recorded every second day as described by Cenci et al. (1998). Cenci M A, Lee C S, Björklund A. L-DOPA-induced dyskinesia in the rat is associated with striatal overexpression of prodynorphin- and glutamic acid decarboxylase mRNA. Eur J Neurosci 1998 10:2694-2706. Briefly, rats are observed individually for 1 min every 10^{th} minute during 3 h following a daily L-DOPA dose. Repetitive movements affecting the side of the body contralateral to the lesion that could not be ascribed to any normal behavioural pattern are classified into four different subtypes: locomotive AIMs, i.e., increased locomotion with contralateral side bias; axial dystonia, i.e., contralateral twisted posturing of the neck and upper body; orolingual AIMs, i.e., stereotyped jaw movements and contralateral tongue protrusion; and forelimb dyskinesia, i.e., repetitive jerks of the contralateral forelimb, sometimes combined with grabbing movements of the paw. Each rat is scored on a severity scale from 0 to 4 based on its frequency and persistence (1=occasional; 2=frequent; 3=continuous but interrupted by sensory distraction; 4=continuous, severe and not interrupted by sensory distraction). The axial, orolingual and forelimb (AOL) AIMs are presented together as a mean (mean AIM score) per time point. In this experiment, L-DOPA (6.25 mg/kg+Benserazide 15 mg/kg) is injected alone or in combination with eltoprazine at 0.08, 0.3, 1.25 and 5 mg/kg.

In order to evaluate the effect of eltoprazine on parkinsonian symptoms and its interaction with L-DOPA, rotational behaviour is evaluated. The numbers of ipsilateral and contralateral turns are recorded in an automated rotometer (TSE Rotameter System, TSE-Systems GmbH, Bad Homburg, Germany) over a period of 180 min. For the effect on parkinsonian symptoms, eltoprazine is administered at 0.3, 1.25 and 5 mg/kg 30 min before the test.

### Statistical Analysis

ANOVA and two-way ANOVA are used to evaluate the significance of the results. Post hoc Tukey test is performed where appropriate.

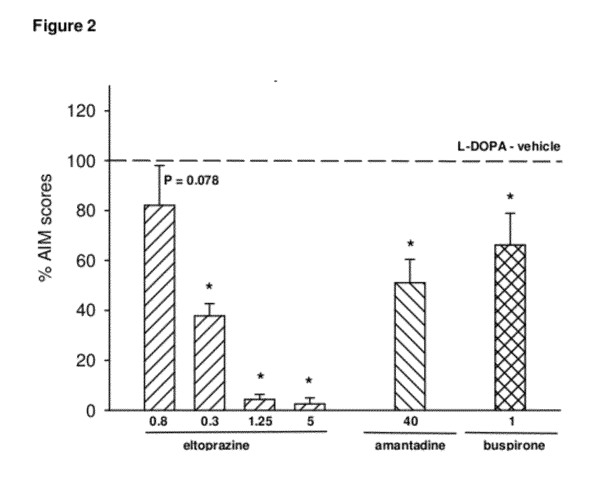

FIG. 2 illustrates the effect of increasing doses of eltoprazine on AIM scores in 6-OHDA-lesioned rats. The data are expressed as % of AIM scores compared to L-DOPA-vehicle-treated animals. * indicates a significant difference with p<0.05 between L-DOPA-vehicle-treated animals (ANOVA).

This result demonstrates that eltoprazine significantly reverses, in a dose-dependent manner, the AIMs that arise in 6-OHDA-lesioned rats after 21 days of treatment with L-DOPA. The effect of amantadine (40 mg/kg) and buspirone (1 mg/kg) in the dyskinetic rat model are presented on the same graph for comparison.

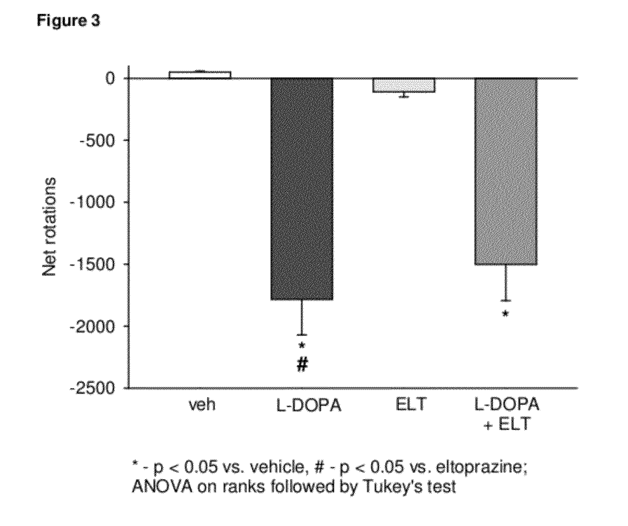

FIG. 3 illustrates the effect of eltoprazine on the turning behaviour induced by high dose of L-DOPA in 6-OHDA-lesioned rats. * indicates a significant difference with p<0.05 versus vehicle-treated animals, # indicates a significant different with p<0.05 versus eltoprazine-treated animals (2-way ANOVA followed by Tukey's Post hoc test).

This result demonstrates that eltoprazine does not interfere with the antiparkinsonian effect of L-DOPA.

### Example 3

### Eltoprazine in in Vivo Models for the Treatment of Anxiety

### Materials and Methods

### Animals

For contextual fear conditioning, open field, fear potentiated startle and elevate plus maze, experimentally naive adult male Sprague-Dawley rats (250-300 g; Janvier, France) were housed in groups of four per cage. Colony room temperature and humidity were maintained respectively at 20±1° C. and 60±3%. Food and water were available ad libitum and the animals were kept under an alternating 12 h/12 h day-night cycle (lights on at 07.00) for at least 6 days before the experiments were started. All experiments were conducted during the light period of the day-night cycle. For the Vogel test, male experimentally naive Wistar rats (180-220 g, State Breeding Farm “Rappolovo”, St. Petersburg, Russia; N=109) were used. Each animal was used only once.

### Drug Treatment

Eltoprazine hydrochloride was dissolved in sterile water. Administration of eltoprazine or vehicle (sterile water) was done s.c. (0.078 to 1.25 mg/kg) once, 60 minutes (30 min in the Vogel test) prior to start of the behavioural assay.

### Contextual Fear Conditioning

For training and testing, subjects were placed in test cages (30 cm wide, 29 cm high and 25 cm deep) equipped with a grid floor consisting of 18 stainless steel bars (3 mm ID). The grid floor is connected to a scrambling shock generator (Model H13-15, Coulbourn Instruments, U.S.A.) by which a 0.45 mA foot shock can be delivered. The complete equipment was placed into a sound-attenuating cubicle (59 cm wide, 61 cm high and 45 cm deep, Coulbourn Instruments, U.S.A). A fan attached on the side wall of the chamber produced a background noise of 60 dB. A noise generator produces additional noise, so that the overall background noise is 65 dB. A digital camera (Model BP 334, Panasonic) is mounted on top of each test cage, adjusted that the total floor of the cage can be observed. The experiment is performed on 2 consecutive days. Conditioning takes place on the first day. Subjects are placed in the test cages, after a 2 min adaptation period, 2 footshocks (0.45 mA, duration 1 s, 60 s inter shock interval) are presented. 30 s after the last footshock, the animals are placed back to their home cages. 24 h after conditioning, subjects are placed back in the test cages and freezing is recorded for a period of 5 min. Freezing is defined as the absence of any movements except those required for respiration. Percent of freezing and total freezing time is taken for statistical analysis.

### Statistical Analysis

Percent of freezing during the 5 min expression test on day 2 is taken for statistical analysis. Data are analysed by one-way ANOVA on ranks followed by rank sum tests.

### Open Field

Locomotor activity was measured in four perspex boxes (ENV-515-16, 43.2 cm×43.2 cm×30 cm) placed in a noise-proof chamber equipped with ventilator and a source of white light (5.6 W) placed 55 cm above floor. Med-Associates Inc. system was used for the measurement of activity. 4 arrays of 16 infrared photo beams placed 3 cm above the floor measured horizontal activity. For the measurement of vertical activity, additional 2 sets of 16 photo beams were placed 15 cm above the floor. The output from the counters was integrated and analysed on-line by an PC computer. Distance travelled was used in further analysis as a measures of locomotion. At least for one hour prior to testing rats were acclimated to the testing room. The recording started immediately after placing animals in the open field and continues for 30 min (6×5 min periods) or 120 min (6×20 min periods).

### Statistical Analysis

Total distance travelled during the 60 min was taken for statistical analysis. Data are analysed by one-way ANOVA followed by Dunnett's test.

### Elevated Plus Maze

The elevated plus maze, made from black polypropylene, consists of two open arms (50.8 cm×10.2 cm surrounded by 1.3 cm walls) and two enclosed arms (50.8×10.2 cm and 40.6 cm high walls), which extended form central platform (10.2×10.2 cm). The maze is elevated 72.4 cm above the floor and it is illuminated by 20 W bulb localised 33 cm above each open arm. Breaks of photo beam are recorded and analysed automatically using an IBM-PC running MED-PC software version IV (Med Associates). A camera hanged 2 m above the maze allows to observe the rat's behaviour in adjacent room where the monitor is placed. Animals are brought to the experimental room 1 h before the start of the experiment. After the injection of testing compound/vehicle rat is placed in the centre of the plus-maze facing an enclosed arm. During the following 5 min period the number of entries into closed and open arms as well as time spent in each type of arm is recorded. An arm entry is recorded when all four paws entering the arm. The obtained results are expressed as mean±S.E.M. percent entries or percent time spent in open arms. The maze is cleaned with water after each trial.

### Statistical Analysis

Percent of time spent in open arm are taken for statistical analysis. Data are analysed by one-way ANOVA followed by Dunnett's test.

### Shock-Suppressed Drinking (Vogel) Test

The apparatus consisted of 4 identical standard operant chambers with grid metal floor. Water bottle spout was protruded through the wall and connected to a shock device. Each chamber was connected with a computer through an interface and controlled by MED-PC software (MED Associates Inc., East Fairfield, Vt., USA). On Day 1, rats were allowed to explore the test chamber for 10 min without shock stimuli to adapt to the test environment. Then animals were water deprived for 24 h. On Day 2, they were placed into the test chambers for baseline measurements. Latency to start drinking was registered, and total drinking time and the licks number were recorded during 5 min timed from the first lick of the bottle spout. After session animals were allowed to drink water for 20 min in their home cage. The middle drinking time in our preliminary experiments was about 140 s. On the base of obtained baseline data, we screened the animals, and underwent to test only subjects which showed drinking time not less than half of middle value, i.e. 70 s (to avoid an interference of possible anxiogenic effect with low motivation to drink). On the test day (Day 3), rats were treated by saline, vehicle or test drug and 30 min later were placed into the chamber. After 10 s of drinking, each subsequent 3 s cumulative drinking was punished by an electric shock (0.2 mA for 0.2 s). The shock intensity and timing were chosen on the basis of preliminary experiments establishing a level of responding that would allow to detect anxiolytic as well as anxiogenic drug effect, i.e. increase and decrease in the number of shocks obtained during the session.

Shock threshold test General procedure was identical to the procedure of shock-suppressed drinking experiment except for the following details. On Day 3 after an initial 10 s of drinking, further drinking was punished by increased shock stimuli from 0.08 mA in 0.03 mA steps (duration 0.2 s, interstimuli interval 5 s). The shock threshold was registered as a visible flinching response of the animal head by an observer blind to the drug treatment.

### Results and Discussion

Acute treatment with eltoprazine revealed controversial effects in different animal models for anxiety. In contextual fear conditioning, eltoprazine dose-dependently induced anxiolytic-like effects as revealed by reduction of the time spent freezing during re-exposure to the conditioning context. Results on locomotor activity in the open field revealed that the observed effects are specific for anxiolytic-like activity and not related to non-specific increase in locomotor activation at anxiolytic doses. In the elevated plus maze test, eltoprazine reduced the time spent in the open arms at the doses of 0.3125 and 1.25 mg/kg, indicating an anxiogenic-like effect. The latter results confirm previous data also demonstrating anxiogenic-like effects of eltoprazine in the same model (Rodgers et al., 1992; Rocha et al., 1994). Importantly, conflicting results have been reported for approved drugs for the treatment of anxiety, e.g. buspirone. While some studies revealed anxiogenic-like effects in the elevated plus maze test (File and Andrews 1991; Collinson and Dawson 1997), another study showed no effect of buspirone in this model (Pellow and File, 1986). In contrast, buspirone has been shown to induce anxiolytic effects in the elevated plus maze (Grundman et al., 2007). These findings suggest that the elevated plus maze may have a rather limited predictability for the clinical situation, at least for serotonergic approaches. In addition, eltoprazine was tested in fear potentiated startle, another preclinical model for anxiety. Acute treatment with eltoprazine revealed an anxiolytic-like effect in one experiment, while a strong trend was observed in a repetition study using the same doses. These data further support the anxiolytic-like potential of the compound. In the shock-suppressed drinking (Vogel) test, eltoprazine showed a tendency to increase the number of accepted punishments, thus indicating a decrease of anxiety. However, it cannot yet been finally ruled out that this was due to, or at least partially influenced by, a decreased sensitivity of the rats to electrical shocks.

In conclusion, eltoprazine produced an anxiogenic-like effect in plus maze, i.e. in a conflict test, confirming previously published data. However, this effect appears to be test-specific, since surprisingly, not anxiogenic, but anxiolytic effects can be seen in other conflict tests. In the Open Field Test, the total distance travelled was significantly increased at a concentration of 1.25 mg/kg, and in the Vogel test, a trend for anxiolytic activity was observed. While the experiment has not yet shown statistically significant results, it did at least not show any anxiogenic activity. In a conditioning test, i.e. contextual fear conditioning, eltoprazine showed very strong anxiolytic effect. In fear potentiated startle, significant effect could be shown in one experiment, while a trend could be found in the other experiment.

In summary, the data from conditioning tests demonstrated that eltoprazine has potential as anxiolytic drug.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

1-14. (canceled)

15. A method for treating or preventing an anxiety disorder in a subject in need thereof, comprising administering to the subject an effective amount of eltoprazine or a pharmaceutically acceptable salt thereof, wherein such anxiety disorder is characterized by a down-regulation of 5-HT1a receptor and/or 5-HT1a receptor activity.

16. The method of claim 15, wherein the eltoprazine is eltoprazine hydrochloride.

17. The method of claim 15, wherein eltoprazine is administered in a range selected from about 5 mg to about 75 mg/day, or in a range from about 5 mg to about 60 mg/day, about 5 mg to about 50 mg/day, about 5 mg to about 40 mg/day, or in a range from about 5 mg to about 20 mg/day, and about 5 mg to about 15 mg/day.

18. The method of claim 17, wherein eltoprazine is administered in a range from about 10 mg to about 15 mg/day.

19. The method of claim 17, wherein eltoprazine is administered at a dose selected from about 5 mg/day, about 10 mg/day, about 15 mg/day, about 20 mg/day, about 40 mg/day, and about 60 mg/day.

20. The method of claim 15, wherein eltoprazine or a pharmaceutically acceptable salt thereof is administered once a day, twice a day (b.i.d.), or three times a day.

21. The method of claim 20, wherein eltoprazine or a pharmaceutically acceptable salt thereof is administered twice a day (b.i.d.).

22. The method of claim 21, wherein the twice daily administered (b.i.d.) is split into a first dose of about 55 to 65% of the total daily dosage amount, and the second dose comprising the remaining total daily dosage amount.

23. The method of claim 21, wherein the second dosage is administered at about lunchtime.

24. The method of claim 15, wherein the subject to be treated suffers from an anxiety disorder, wherein such disorder is characterized by a down-regulation of 5-HT1a receptor and/or 5-HT1a receptor activity.

25. The method of claim 24, wherein the subject suffers from posttraumatic stress disorder.

26. The method of claim 15, wherein the eltoprazine is administered in the form of an oral formulation.
